(19) United States
(12) Patent Application Publication
(10) Pub. No.: US 2022/0300699 A1
(43) Pub. Date: Sep. 22, 2022

(54) COMBINATION THERAPY TO TREAT CANCER AND USES THEREOF

(71) Applicant: ACROSS SYSTEMS GMBH, Karlsbad (DE)

(72) Inventors: Boyang WU, Pullman, WA (US); Baron BECHTOLD, Pullman, WA (US); Karen VO, Pullman, WA (US); Tyler BLAND, Pullman, WA (US)

(21) Appl. No.: 17/599,618

(22) PCT Filed: Mar. 27, 2020
(86) PCT No.: PCT/EP2020/025102

§ 371 Date: Sep. 29, 2021

Related U.S. Application Data
(60) Provisional application No. 62/826,626, filed on Mar. 29, 2019.

(51) Int. Cl. G06F 40/166; G06F 3/0488; G06F 3/0484; G06F 40/279; G06F 40/49

(52) U.S. Cl. CPC G06F 40/166; G06F 3/0488; G06F 3/0484; G06F 40/279; G06F 40/49

(57) ABSTRACT

The present invention discloses a method for amending translated draft text, an electronic device for amending translated draft text, and an APP implementing said method. The present invention provides an editing function for machine translation text which issues a final feedback or approval.

### BACKGROUND OF THE INVENTION

### Field of the Invention

The present invention relates to a method for user to edit machine translation text, and more particularly to a method for amending translated draft text, an electronic device implementing said method and a recording medium implementing said method.

### Description of Prior Art

Machine aided translation is becoming popular with the development of computer networking and smartphones. The post edit for the machine translation text is now becoming possible with electronic devices.

Via the webpage, the Google Translator does provide an option for a user to click on the target sentence so that the user is able to input his/her suggestion if he is not satisfied with the target sentence. With the pen icon on the webpage, the user can make changes to the sentences to correct them. However, the Google Translator only allows to change the full sentence, but not single specific word(s). Furthermore, there is no suggestion of alternatives or options for the user and hence no click-interaction rather than merely typing the text manually. In other words, the Google Translator only provides a function for one-time user input for suggestion, but no further presenting any feedback or receiving additional approval.

Meanwhile, the DeepL Translator also provides a function for one-time user input. Currently the DeepL Translator DeepL could take the input from the user and adjust or edit the sentence of machine translation text. However, there is no explicit final feedback or approval either from the same user himself or other user(s).

### Problem to Be Solved

As mentioned above, since users regularly cannot receive any review or approval for his editing, the quality of the machine-translation based text is not satisfactory.

Therefore, it is an object of the present invention to provide that editing function for machine translation text which issues a final feedback or approval.

### SUMMARY OF THE INVENTION

### Solution for the Problem

This problem is solved by creating performing a procedure for amending the draft text, until a user approval for the amended text is received.

The present disclosure provides a method for amending translated draft text, said method applied in an electronic device comprising a touch screen and a display. The method comprising: step of receiving and presenting (50) translated draft text via the display; determining (50a) whether or not user approval for the translated draft text is received from the electronic device. When the user approval for the draft text is received from the electronic device, the method comprising: step of outputting the draft text.

Otherwise, when the user approval for the draft text is not received from the electronic device, a procedure for amending the draft text is performed, until a user approval for an amended text is received from the electronic device. The procedure comprising: step of receiving (60) a first user input for selecting at least one word in the translated draft text via the touch screen of the electronic device; step of presenting (70) at least one alternative for replacing the selected word via the display; step of receiving (80) a second user input for choosing the alternative replacing the selected word via the touch screen; step of replacing (90) the selected word with the alternative and presenting the amended text; and step of determining (90a) whether or not the user approval for the amended text is received from the electronic device. When a user approval for an amended text is received from the electronic device, the method comprising: step of outputting the amended text.

The present disclosure provides a method for translating and amending translated draft text. The method comprising: step of receiving (10) input text for translation; step of analyzing (20) the input text for terminology and subject-matter; step of translating (30) the input text into translated draft text using terminology database; step of dividing (40) the translated draft text into translated draft sentences; step of receiving and presenting (50) the translated draft sentence via a display of an electronic device; determining (50a) whether or not user approval for the translated draft text is received from the electronic device. When the user approval for the draft text is received from the electronic device, the method comprising: step of outputting the draft text.

Otherwise, when the user approval for the draft text is not received from the electronic device, a procedure for amending the draft text is performed, until a user approval for an amended text is received from the electronic device. The procedure comprising: step of receiving (60) a first user input for selecting at least one word in the translated draft text via the touch screen of the electronic device; step of presenting (70) at least one alternative for replacing the selected word via the display; step of receiving (80) a second user input for choosing the alternative replacing the selected word via the touch screen; step of replacing (90) the selected word with the alternative and presenting the amended text; and step of determining (90a) whether or not the user approval for the amended text is received from the electronic device. When a user approval for an amended text is received from the electronic device, the method comprising: step of outputting the amended text.

The present disclosure provides an electronic device for amending translated draft text, said electronic device comprising a display, a touch screen, a processor and a memory in which a computer program is stored. The processor is configured to: receive translated draft text and present the draft text via the display, determine whether or not user approval for the translated draft text is received from the electronic device. When the user approval for the draft text is received from the electronic device, the processor is configured to output the draft text.

Otherwise, when the user approval for the draft text is not received from the electronic device, the processor is further configured to perform a procedure for amending the draft text, until a user approval for the amended text is received from the electronic device. The procedure comprising: receiving a first user input for selecting at least one word in the translated draft text via the touch screen; presenting at least one alternative for replacing the selected word via the display; receiving a second user input for choosing the alternative replacing the selected word via the touch screen; replacing the selected word with the alternative and presenting the amended text; and determining whether or not the user approval for the amended text is received from the electronic device. When a user approval for an amended text is received from the electronic device, the processor is configured to output the amended text.

The present disclosure provides a computer program product comprising instructions which, when the program is executed by a computer, cause the computer to carry out the steps of any one of the above methods.

The present disclosure provides a recording medium readable by a computer and having recorded thereon a computer program including instructions for executing the steps of any one of the above methods.

### BRIEF DESCRIPTION OF DRAWING

Many aspects of the embodiments can be better understood with references to the following drawings. The components in the drawings are not necessarily drawn to scale, the emphasis instead being placed upon clearly illustrating the principles of the embodiments. Moreover, in the drawings, like reference numerals designate corresponding parts throughout two views.

The invention itself may be best understood by reference to the following detailed description of the invention, which describes an exemplary embodiment of the invention, taken in conjunction with the accompanying drawings, in which:

FIGS. 1.1-1.4 are schematic views of a transition of a display of a mobile device, illustrating the method for amending translated draft text according to a first embodiment of the present invention;

FIGS. 2.1-2.4 are schematic views of a transition of a display of a mobile device, illustrating the method for amending translated draft text according to a second embodiment of the present invention;

FIGS. 3.1-3.4 are schematic views of a transition of a display of a mobile device, illustrating the method for amending translated draft text according to a third embodiment of the present invention;

FIGS. 4.1-4.4 are schematic views of a transition of a display of a mobile device, illustrating the method for amending translated draft text according to a fourth embodiment of the present invention;

FIG. 5 is a schematic view for user account setting according to a fifth embodiment of the present invention;

FIG. 6 is a flow chart for a method for translating and amending translated draft text according to a sixth embodiment of the present invention; and

FIG. 7 is a schematic view for a method for amending translated draft text operating in each of multiple smartphones according to embodiments of the present invention.

### DETAILED DESCRIPTION OF THE INVENTION

The present disclosure will be further described in detail below with reference to the drawings and specific embodiments, in order to better understand the objective, the technical solution and the advantage of the present disclosure. It should be understood that the specific embodiments described herein are merely illustrative and are not intended to limit the scope of the disclosure.

It also should be understood that the specific terms in the present disclosure are inter-changeable terms, for examples, “adjust”, “amend”, “correct” or “edit” can be inter-changeably used for the post editing machine translation text; also “user equipment” or “electronic device” or “mobile device” can be inter-changeably used. The graphical user interface is used to amend the translated text. This amendment includes but is not limited to editing and correction.

It further should be understood that the method can be implemented by application or APP on smart phones. The smart phone APP is used to run program, to display information and to communicate with backend or servers.

It further should be understood that the “user” should be interpreted in its broadest meaning. A user could be an end user (e.g., in the above examples Google Translator or DeepL Translator), or an editor for post-translation text (i.e., a post-editor).

Reference will now be made to the drawing figures to describe the present invention in detail.

### Embodiment 1

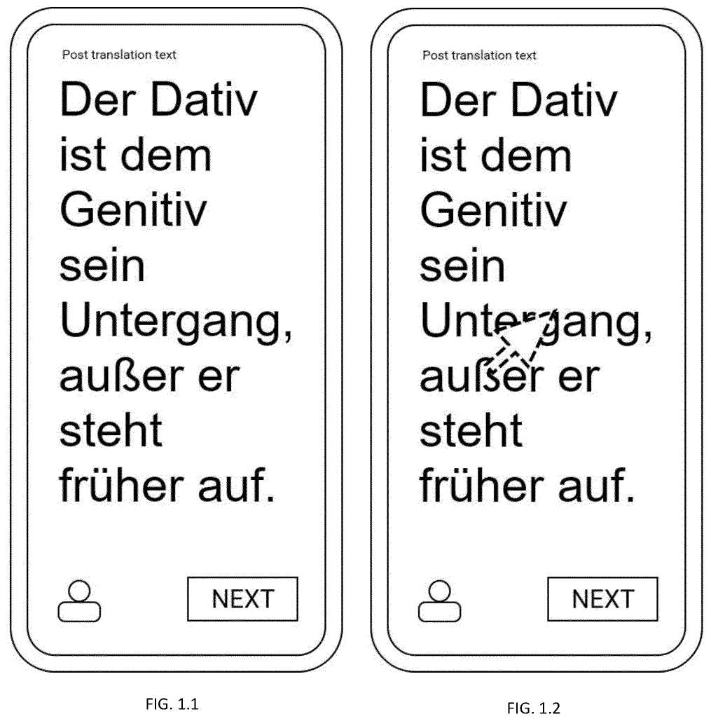

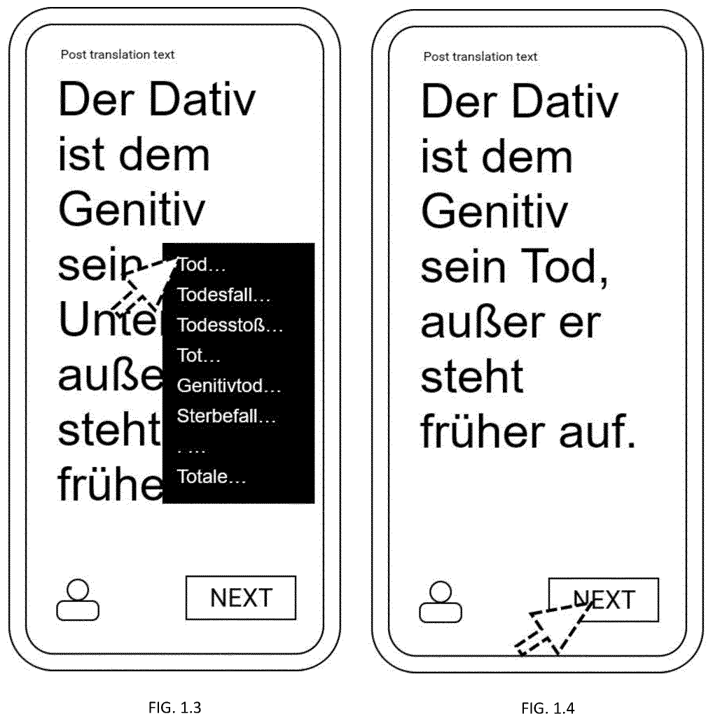

As shown in FIGS. 1.1-1.4, a method is introduced for amending translated draft text with a graphical user interface (GUI) of mobile devices. Specifically, FIG. 1.1 shows a display of a mobile device, illustrating (STEP 50) the received draft text (i.e., post-translation text) to be amended.

Furthermore, after the step of receiving draft text, it is determined (STEP 50a) whether or not user approval for the draft text is received from the mobile device. If the user approval for the draft text is not received from the mobile device, the following procedure is performed. Otherwise, the draft text is outputted, for example to the backend or server.

As shown in FIGS. 1.2-1.4, a procedure is introduced to be performed within the method. The procedure is performed for amending the draft text, until a user approval for the amended text is received from the mobile device.

When a first user input for selecting at least one word in the draft text is received (STEP 60) via a touch screen of the mobile device, a prompt icon (e.g., an arrow) is shown to be located at the selected word in the draft text. See FIG. 1.2, the word “Untergang” is selected as an example. The prompt icon provides (STEP 70) the user with a prompt for at least one alternative or an option for replacing the selected word, so as to amend the selected word. See FIG. 1.3, a list of multiple alternatives or options is shown, for example, “Tod”, “Todesfall”, “Tot”, “Totale”, etc., which can be selected for replacing the word “Untergang”. A second user input is received (STEP 80) via the touch screen for choosing the one alternative, in order for replacing the selected word “Untergang”. Based on the chosen alternative, the selected word “Untergang” is replaced (STEP 90) with the alternative word and presented within the amended text. See FIG. 1.4, the alternative word “Tod” is chosen for replacing the word “Untergang”. By clicking the “NEXT” button, the amended text is sent for a further user approval.

If a user approval is not received from the mobile device (STEP 90a), the procedure is repeated. If a user approval is received from the mobile device (STEP 90a), the procedure ends and the amended text is outputted, for example to the backend or server.

It could be understood that the user approval for the translated draft and the user approval for the amended text is received via the touch screen of the mobile device.

Furthermore, the translated draft text is a sentence, and the procedure is based on a draft sentence.

It should be understood that other views for the procedure repetition are similar to FIGS. 1.2-1.4 in the sense of the inventive concept and thus are omitted.

### Embodiment 2

Embodiment 2 is used to show a reward or bonus for his input option after the user approval.

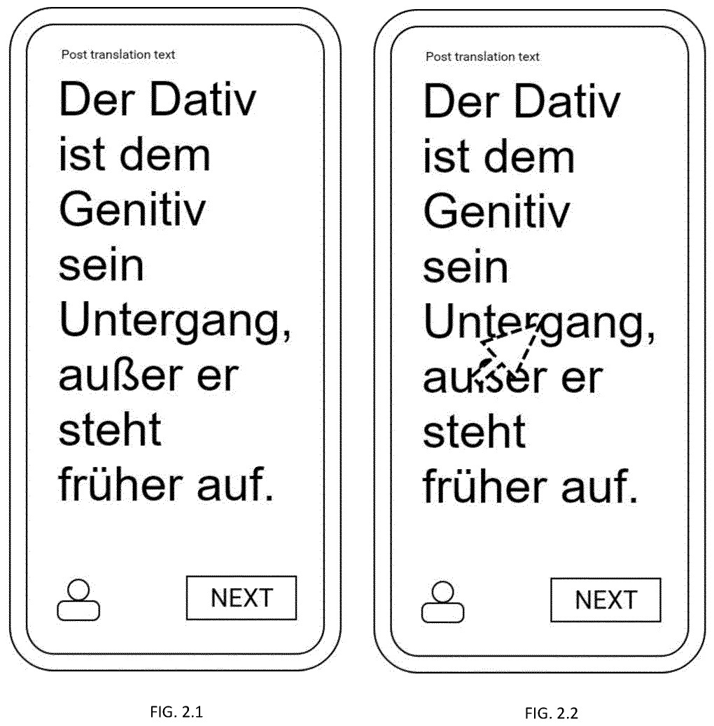

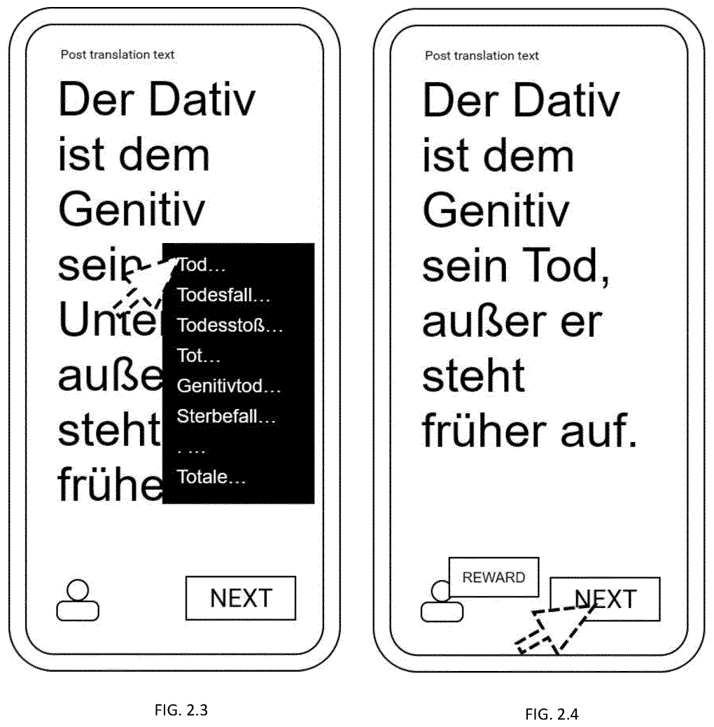

Specifically, as shown in FIG. 2.4, the reward or bonus is shown on the bottom of the display page. Further, as shown in FIG. 5, a user account setting is presented.

It should be understood that other details for the procedure repetition are similar to Embodiment 1 in the sense of the inventive concept and thus will not repeated here.

### Embodiment 3

Embodiment 3 is used to simultaneously show on the same display page the translated draft text in the original language (or the source language) and the amended text in the target language. Further, the embodiment 3 is used to show the reward or bonus after the user inputs the prompted option.

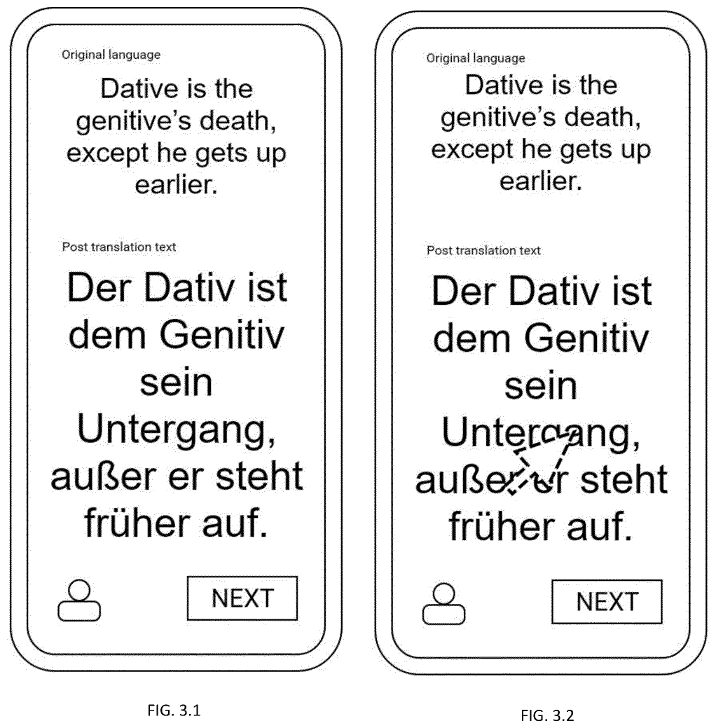

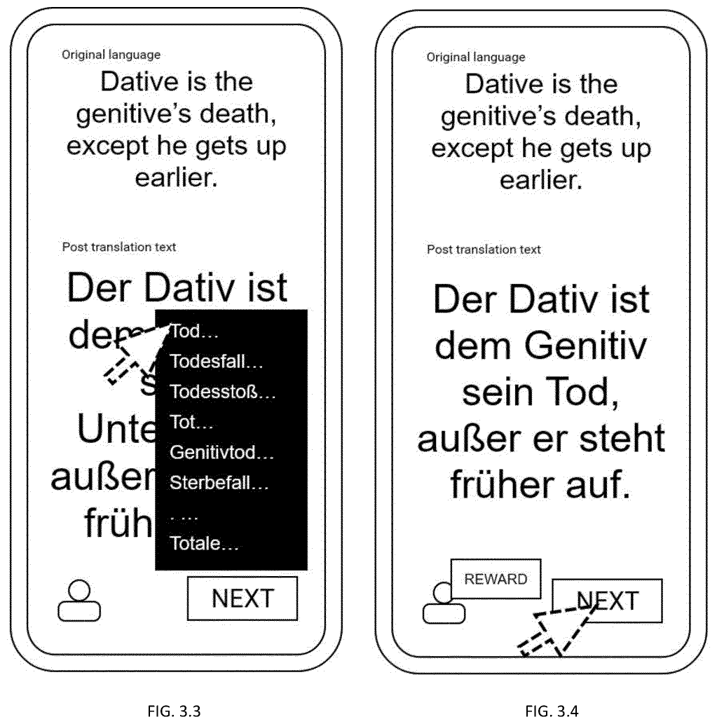

Specifically, as shown in FIGS. 3.1-3.4, the draft text is shown on the upper part of the display page, while the amended text is shown on the lower part of the same display page.

Further, as shown in FIG. 3.4, the reward or bonus is shown on the bottom of the display page.

It should be understood that other details for the procedure repetition are similar to Embodiment 1 in the sense of the inventive concept and thus will not repeated here.

### Embodiment 4

Embodiment 4 is used to simultaneously show on the same display page the translated draft text in the original language and the amended text in the target language. Further, the reward or bonus is not shown in the embodiment 4.

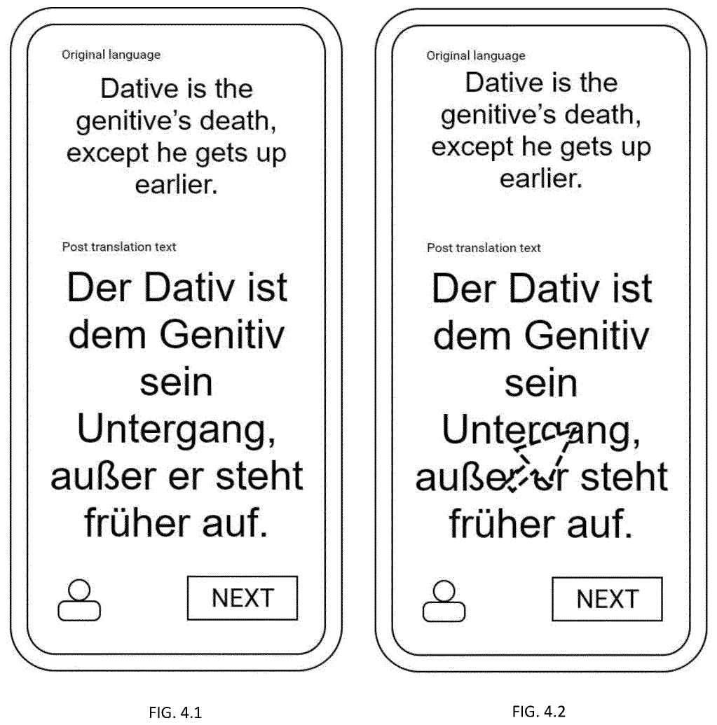

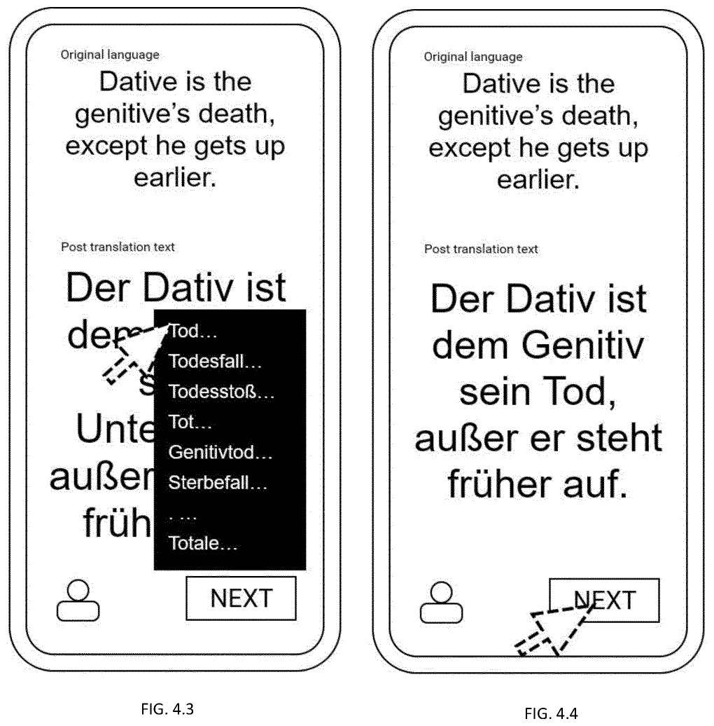

Specifically, as shown in FIGS. 4.1-4.4, the draft text is shown on the upper part of the display page, while the amended text is shown on the lower part of the same display page.

It should be understood that other details for the procedure repetition are similar to Embodiment 1 in the sense of the inventive concept and thus will not repeated here.

### Embodiment 5

As shown in FIG. 5, an electronic device is introduced for amending translated draft text. The electronic device comprises a display, a touch screen, a processor (not shown) and a memory (not shown) in which a computer program is stored. The processor is configured to receive and present translated draft text via the display; and to determine whether or not user approval for the translated draft text is received from the electronic device. The processor is further configured to perform the procedure for amending the draft text, until a user approval for the amended text is received from the electronic device.

It should be understood that details for the procedure repetition are according to Embodiment 1 in the sense of the inventive concept and thus will not repeated here.

Moreover, FIG. 5 also illustrates a user account setting displayed with a graphical user interface (GUI) of the electronic device. Specifically, under a virtual user account “Max Mustermann”, multiple entries of his amendment(s) associated with this user account are displayed with respective progress status.

In a history record area, status for each entry (related to the translated draft text or sentence) is labelled with progress information. For example, if the amendment is completed for one entry, this entry is marked as “OK”, while other entries for which amendment still under work are marked as “NOT OK”.

The marks are determined by the feedback of the sequentially next user or editor to whom the sentence after the current amendment will be shown. If the next user does approve the sentence without further edit, it is set to “OK”. If the next user changes the sentence, it's set as “NOT OK”. Additionally, for every mark “OK”, the user level will increase; for every mark “NOT OK”, the user level will decrease.

### Embodiment 6

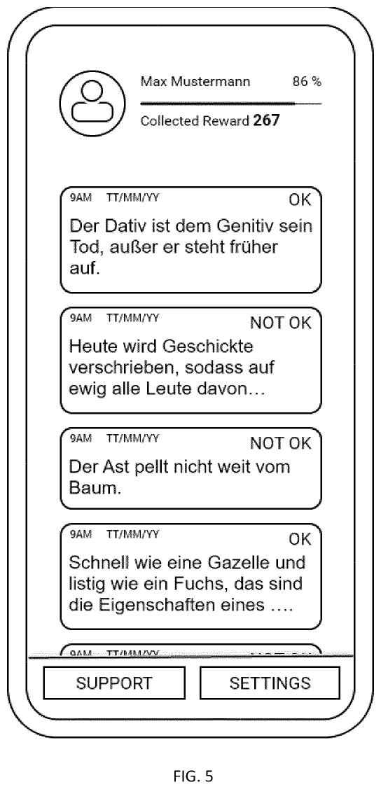

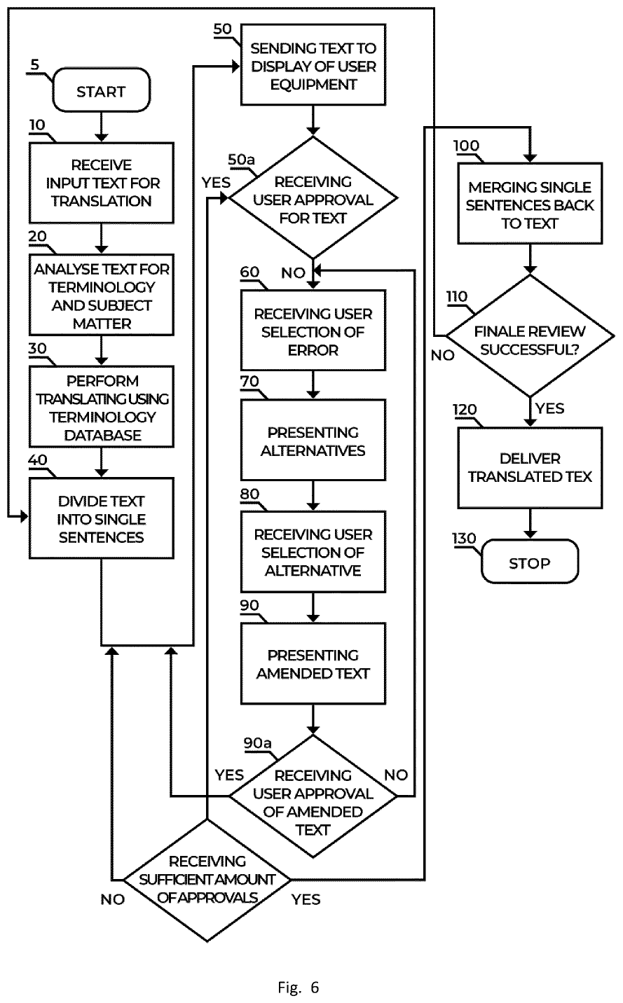

As shown in FIG. 6, a method is introduced for translating and amending translated draft text. Specifically, input text in the original language (or the source language) for translation is received (STEP 10). The input text is analyzed (STEP 20) in respect to terminology and subject-matter, for example by the backend. By using terminology database, the input text is translated (STEP 30) into translated draft text in the target language. Then the translated draft text is divided (STEP 40) into translated draft sentence(s).

The draft sentence (i.e., post-translation sentence) is received by the user and illustrated (STEP 50) for amendment. Further, after the step of receiving draft text, it is determined (STEP 50a) whether or not user approval for the draft text is received.

If the user approval for the draft text is received from the electronic device, the draft text is outputted, for example to the backend or server.

If the user approval for the draft text is not received from the electronic device, the following procedure is performed. When a first user input for selecting at least one word in the draft text is received (STEP 60), a prompt icon is shown to be located at the selected word in the draft text. The prompt icon provides (STEP 70) the user with a prompt for at least one alternative or an option for replacing the selected word, so as to amend the selected word. A second user input is received (STEP 80) via the touch screen for choosing the one alternative, in order for replacing the selected word. Based on the chosen alternative, the selected word is replaced (STEP 90) with the alternative word and presented within the amended text.

Next, the amended text is sent for user approval. If a user approval is not received from the electronic device (STEP 90a), the procedure is repeated. If a user approval is received from the electronic device (STEP 90a), the procedure ends and the amended text is outputted, for example to the backend or server.

It should be understood that detailed examples for the procedure repetition are similar to Embodiment 1 in the sense of the inventive concept and thus are omitted.

### Embodiment 7a

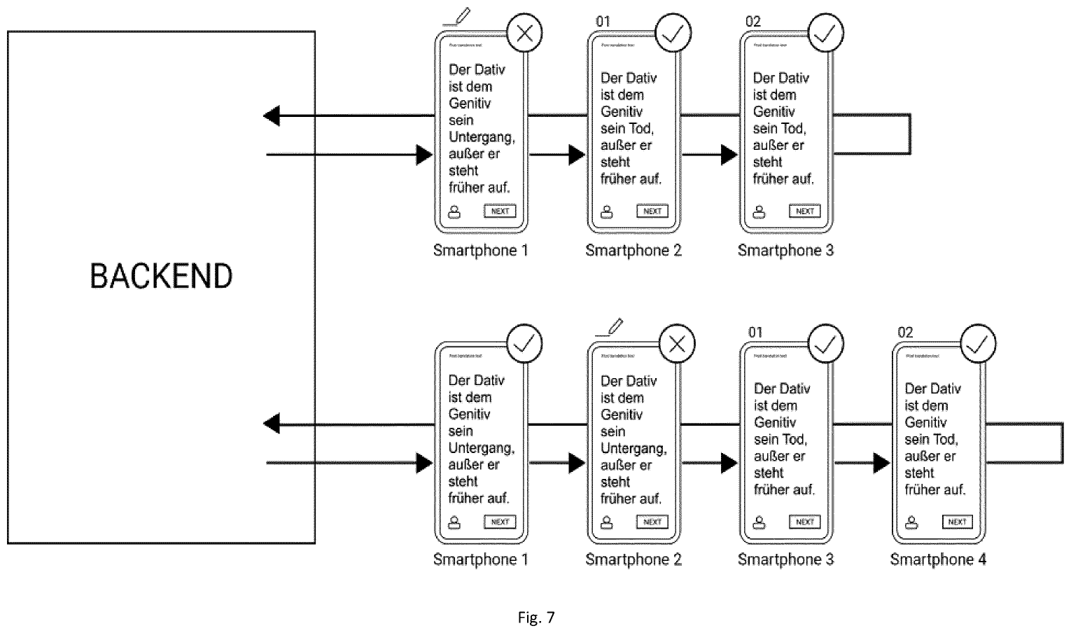

As shown in FIG. 7 (see the upper part), a method for amending translated draft text is operating in each of smartphones 1, 2 and 3. Reference is now made in sequence to each of smartphones 1, 2 and 3, to describe the present embodiment in detail.

Specifically, first of all, the smartphone 1 receives the translated draft text or sentence, for example from the backend. The smartphone 1 determines that a user approval for the draft text is not received from the smartphone 1, for example from its touch screen. See FIGS. 1.2-1.4, a procedure for amending the draft text is performed, where the selected word “Untergang” is replaced with the alternative word “Tod”. The smartphone 1 determines that the user approval for the amended text is received from the smartphone 1, for example from the touch screen, and outputs the amended text.

Secondly, the smartphone 2 receives the amended text, for example from the smartphone 1. The smartphone 2 determines that a user approval for the amended text is received from the smartphone 2, for example from the touch screen. The smartphone 2 can output the amended text without any change.

Thirdly, the smartphone 3 receives the amended text, for example from the smartphone 2. The smartphone 3 determines that a user approval for the amended text is received from the smartphone 3, for example from the touch screen. The smartphone 3 can output the amended text without any change.

### Embodiment 7b

As shown in FIG. 7 (see the lower part), a method for amending translated draft text is operating in each of smartphones 1, 2, 3 and 4. Reference is now made in sequence to each of smartphones 1, 2, 3 and 4, to describe the present embodiment in detail.

Specifically, first of all, the smartphone 1 receives the translated draft text or sentence, for example from the backend. The smartphone 1 determines that a user approval for the draft text is received from the smartphone 1, for example from the touch screen. The smartphone 1 can output the draft text or sentence without any change.

Secondly, the smartphone 2 receives the draft text or sentence text, for example from the smartphone 1. The smartphone 2 determines that a user approval for the draft text is not received from the smartphone 2, for example from its touch screen. See FIGS. 1.2-1.4, a procedure for amending the draft text is performed, where the selected word “Untergang” is replaced with the alternative word “Tod”. The smartphone 2 determines that the user approval for the amended text is received from the smartphone 2, for example from the touch screen, and outputs the amended text.

Thirdly, the smartphone 3 receives the amended text, for example from the smartphone 2. The smartphone 3 determines that a user approval for the amended text is received from the smartphone 3, for example from the touch screen. The smartphone 3 can output the amended text without any change.

Fourthly, the smartphone 4 receives the amended text, for example from the smartphone 3. The smartphone 4 determines that a user approval for the amended text is received from the smartphone 4, for example from the touch screen. The smartphone 4 can output the amended text without any change.

It should be understood that the above detailed examples for the procedure repetition are similar to Embodiment 1 in the sense of the inventive concept and thus are omitted.

While the disclosure has been described by way of example and in terms of exemplary embodiment, it is to be understood that the disclosures is not limited thereto. It is to be understood that the above-described embodiments are merely illustrative and not restrictive. To the contrary, it is intended to lamp shade various modifications and similar arrangements (as would be apparent to those skilled in the art).

It will be readily understood by those skilled in the art that the above various preferred embodiments can be freely combined and superimposed without conflict. Various obvious or equivalent modifications or alterations to the above-described details will be included in the scope of the claims of the present disclosure without departing from the basic principles of the application. Therefore, the scope of the appended claims should be accorded the broadest interpretation so as to encompass all such modifications and similar arrangements.

1. A method for amending translated draft text, said method applied in an electronic device comprising a touch screen and a display, said method comprising:
step of receiving and presenting (50) translated draft text via the display;
determining (50a) whether or not user approval for the translated draft text is received from the electronic device;
when the user approval for the draft text is not received from the electronic device,
performing a procedure for amending the draft text, until a user approval for an amended text is received from the electronic device, said procedure comprising:
step of receiving (60) a first user input for selecting at least one word in the translated draft text via the touch screen of the electronic device;
step of presenting (70) at least one alternative for replacing the selected word via the display;
step of receiving (80) a second user input for choosing the alternative replacing the selected word via the touch screen;
step of replacing (90) the selected word with the alternative and presenting the amended text; and
step of determining (90a) whether or not the user approval for the amended text is received from the electronic device;
step of outputting the amended text;
or
when the user approval for the draft text is received from the electronic device, step of outputting the draft text.

2. The method in claim 1, wherein said user approval for the translated draft and the user approval for the amended text is received via the touch screen.

3. The method in claim 1 or 2, wherein the translated draft text is a sentence, and said procedure is based on sentence.

4. A method for translating and amending translated draft text, comprising:
step of receiving (10) input text for translation;
step of analyzing (20) the input text for terminology and subject-matter;
step of translating (30) the input text into translated draft text using terminology database;
step of dividing (40) the translated draft text into translated draft sentences;
step of receiving and presenting (50) the translated draft sentence via a display of an electronic device;
determining (50a) whether or not user approval for the translated draft text is received from the electronic device;
when the user approval for the draft text is not received from the electronic device,
performing a procedure for amending the draft sentence, until a user approval for an amended text is received from the electronic device, said procedure comprising:
step of receiving (60) a first user input for selecting at least one word in the translated draft sentence via a touch screen of the electronic device;
step of presenting (70) at least one alternative for replacing the selected word via the display;
step of receiving (80) a second user input for choosing the alternative replacing the selected word via the touch screen;
step of replacing (90) the selected word with the alternative and presenting the amended sentence;
step of determining (90a) whether or not the user approval for the amended sentence is received from the electronic device;
step of outputting the amended text;
or
when the user approval for the draft sentence is received from the electronic device, step of outputting the draft sentence.

5. An electronic device for amending translated draft text, said electronic device comprising a display, a touch screen, a processor and a memory in which a computer program is stored,
wherein the processor is configured to:
receive translated draft text and present the draft text via the display,
determine whether or not user approval for the translated draft text is received from the electronic device;
characterised in that when the user approval for the draft text is not received from the electronic device, the processor is further configured to perform a procedure for amending the draft text, until a user approval for the amended text is received from the electronic device, said procedure comprising:
receiving a first user input for selecting at least one word in the translated draft text via the touch screen;
presenting at least one alternative for replacing the selected word via the display;
receiving a second user input for choosing the alternative replacing the selected word via the touch screen;
replacing the selected word with the alternative and presenting the amended text; and
determining whether or not the user approval for the amended text is received from the electronic device;
the processor is configured to output the amended text,
or
when the user approval for the draft text is received from the electronic device, the processor is configured to output the draft text.

6. The electronic device in claim 5, wherein said user approval for the translated draft and the user approval for the amended text is received via the touch screen.

7. The electronic device in any one of the preceding claims 5-6, wherein the translated draft text is a sentence, and said procedure is based on sentence.

8. A computer program product comprising instructions which, when the program is executed by an electronic device or a computer, cause the computer to carry out the steps of the method according to any one of claims 1 to 4.

9. A recording medium readable by an electronic device or a computer and having recorded thereon a computer program including instructions for executing the steps of a method according to any one of claims 1 to 4.
